(19) United States
(12) Patent Application Publication
(10) Pub. No.: US 2026/0053555 A1
(43) Pub. Date: Feb. 26, 2026

(54) SYSTEM AND METHODS FOR TISSUE VAPORIZATION USING A SURROUNDING NON-CONDUCTIVE FLUID

(71) Applicant: Boston Scientific Scimed, Inc., Maple Grove, MN (US)

(72) Inventors: Christian BALKOVEC, Cambridge (CA); Gareth DAVIES, Toronto (CA)

(21) Appl. No.: 19/271,104

(22) Filed: Jul. 16, 2025

Related U.S. Application Data
(60) Provisional application No. 63/685,666, filed on Aug. 21, 2024.

(51) Int. Cl. A61B 18/14; A61B 18/00; A61B 18/12

(52) U.S. Cl. CPC A61B 18/1492; A61B 18/1206; A61B 2018/00357; A61B 2018/00625

(57) ABSTRACT

An electrosurgical system for piercing tissue includes an electrosurgical generator configured to generate radiofrequency (RF) energy, a fluid system configured to deliver electrically insulating fluid, and a crossing member connected to the electrosurgical generator and the fluid system. The crossing member includes an electrode positioned at a distal tip of the crossing member, and a first fluid port positioned adjacent to the electrode, the first fluid port configured to direct the electrically insulating fluid around the electrode.

### CROSS REFERENCE TO RELATED APPLICATIONS

This application claims priority to U.S. Provisional Application No. 63/685,666 entitled “SYSTEM AND METHODS FOR TISSUE VAPORIZATION USING A SURROUNDING NON-CONDUCTIVE FLUID,” filed Aug. 21, 2024, which is hereby incorporated by reference in its entirety.

### TECHNICAL FIELD

The present disclosure relates to medical systems and methods for vaporizing tissue in a patient. More specifically, the present disclosure relates to medical systems and methods for puncturing bodily tissues such as the atrial septum with an electrode by dielectric breakdown.

### BACKGROUND

Minimally invasive surgical techniques can employ catheters to provide access into a patient's body. Surgical instruments can be routed through the catheters such that the instruments can be positioned where they are needed for a given procedure. For example, in a cardiac procedure involving the left atrium of the heart, it may be advantageous to route a catheter into the right atrium and perform a transseptal puncture procedure to enter the left atrium. Such a transseptal puncture procedure can be performed using one or more electrodes that are passed through a catheter and energized in the radiofrequency (RF) range. While this procedure can vaporize the targeted septal tissue, it can also vaporize liquids (e.g., blood) that are in contact with the electrode(s).

### SUMMARY

In Example 1, An electrosurgical system for piercing tissue, the electrosurgical system comprising: an electrosurgical generator configured to generate radiofrequency (RF) energy; a fluid system configured to deliver electrically insulating fluid; and a crossing member connected to the electrosurgical generator and the fluid system, the crossing member including: an electrode positioned at a distal tip of the crossing member; and a first fluid port positioned adjacent to the electrode, the first fluid port configured to direct the electrically insulating fluid around the electrode.

In Example 2, the electrosurgical system of Example 1, wherein: the crossing member comprises a cover surrounding and spaced apart from a longitudinal portion of the electrode; and the first fluid port is defined by the cover and the electrode.

In Example 3, the electrosurgical system of Example 2, wherein a conductor extends between the electrode and the electrosurgical generator.

In Example 4, the electrosurgical system of Example 3, wherein the conductor includes an exterior insulative layer.

In Example 5, the electrosurgical system of Example 4, wherein the cover comprises a plurality of supports that are in direct contact with the exterior insulative layer.

In Example 6, the electrosurgical system of Example 5, wherein each of the supports has a symmetrical airfoil/hydrofoil shape.

In Example 7, the electrosurgical system of Example 4, wherein the cover comprises a plurality of supports that are in direct contact with the conductor.

In Example 8, the electrosurgical system of Example 7, wherein each of the supports has a symmetrical airfoil/hydrofoil shape.

In Example 9, the electrosurgical system of any of Examples 3-8, wherein the crossing member further comprises a dilator slidably positioned around the conductor.

In Example 10, the electrosurgical system of Example 9, wherein the cover is an integral portion of the dilator.

In Example 11, the electrosurgical system of Example 1, wherein: the crossing member further comprises a lumen connected to the first fluid port and a second fluid port; and the electrode is positioned between the first fluid port and the second fluid port.

In Example 12, the electrosurgical system of any of Examples 1-11, wherein the crossing device is a wire.

In Example 13, the electrosurgical system of any of Examples 1-11, wherein the crossing device is a needle.

In Example 14, the electrosurgical system of any of Examples 1-13, wherein the electrically insulating fluid is dextrose.

In Example 15, the electrosurgical system of any of Examples 1-13, wherein the electrically insulating fluid is carbon dioxide.

In Example 16, an electrosurgical system for piercing tissue, the electrosurgical system comprising: an electrosurgical generator configured to generate radiofrequency (RF) energy; a fluid system configured to deliver electrically insulating fluid; and a crossing member connected to the electrosurgical generator and the fluid system, the crossing member including: an electrode positioned at a distal tip of the crossing member; and a first fluid port positioned adjacent to the electrode, the first fluid port configured to direct the electrically insulating fluid around the electrode.

In Example 17, the electrosurgical system of Example 16, wherein: the crossing member comprises a cover surrounding and spaced apart from a longitudinal portion of the electrode; and the first fluid port is defined by the cover and the electrode.

In Example 18, the electrosurgical system of Example 16, wherein: the crossing member further comprises a lumen connected to the first fluid port and a second fluid port; and the electrode is positioned between the first fluid port and the second fluid port.

In Example 19, the electrosurgical system of Example 16, wherein the electrically insulating fluid is dextrose.

In Example 20, the electrosurgical system of Example 16, wherein the electrically insulating fluid is carbon dioxide.

In Example 21, the electrosurgical system of Example 16, wherein the crossing device is a wire.

In Example 22, the electrosurgical system of Example 16, wherein the crossing device is a needle.

In Example 23, an electrosurgical system for piercing tissue, the electrosurgical system comprising: an electrosurgical generator configured to generate radiofrequency (RF) energy; a fluid system configured to deliver electrically insulating fluid; and a crossing member connected to the electrosurgical generator and the fluid system, the crossing member including: an electrode positioned at a distal tip of the crossing member; and a first fluid port that extends through the electrode, the first fluid port configured to direct the electrically insulating fluid around the electrode.

In Example 24, the electrosurgical system of Example 6, wherein the first fluid channel is one of an array of fluid channels positioned around the electrode but not at the distal tip.

In Example 25, the electrosurgical system of Example 23, wherein the crossing device is a wire.

In Example 26, the electrosurgical system of Example 23, wherein the crossing device is a needle.

In Example 27, the electrosurgical system of Example 23, wherein the electrically insulating fluid is dextrose.

In Example 28, the electrosurgical system of Example 23, wherein the electrically insulating fluid is carbon dioxide.

In Example 29, an electrosurgical system for piercing tissue, the electrosurgical system comprising: an electrosurgical generator configured to generate radiofrequency (RF) energy; a fluid system configured to deliver electrically insulating fluid; and a crossing member connected to the electrosurgical generator and the fluid system, the crossing member including: an electrode positioned along an exterior of the crossing member proximal from a distal tip; and a first fluid channel that extends through an interior of the crossing member, the first fluid channel being fluidly connected to a fluid port configured to direct the electrically insulating fluid around the electrode.

In Example 30, the electrosurgical system of Example 29, wherein the distal tip of the crossing member is configured to puncture tissue.

In Example 31, the electrosurgical system of Example 29, wherein the first fluid channel is one of an array of fluid channels positioned around the electrode.

In Example 32, the electrosurgical system of Example 29, wherein the crossing device is a wire.

In Example 33, the electrosurgical system of Example 29, wherein the crossing device is a needle.

In Example 34, the electrosurgical system of claim 29, wherein the electrically insulating fluid is dextrose.

In Example 35, the electrosurgical system of Example 29, wherein the electrically insulating fluid is carbon dioxide.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example electrosurgical system for treating a patient, such as a heart or the vasculature of a patient, including an electrosurgical generator, a transseptal crossing member, and an insulating fluid system, consistent with various aspects of the present disclosure.

FIG. 2 is a cutaway view of a heart including an atrial septum and the transseptal crossing system, consistent with various aspects of the present disclosure.

FIG. 3 is a side view of the distal end of the transseptal crossing member, consistent with various aspects of the present disclosure.

FIGS. 4A and 4B are views of a distal end of the transseptal crossing member. More specifically, FIG. 4A is a cross-sectional view of the distal tip of the transseptal crossing member as indicated by line 4-4 in FIG. 4B, consistent with various aspects of the present disclosure. FIG. 4B is a front view of a crossing device, consistent with various aspects of the present disclosure.

FIG. 5 is a flowchart of a method of crossing the atrial septum, consistent with various aspects of the present disclosure.

FIGS. 6A and 6B are a series of schematic diagrams of operations of crossing the atrial septum according to the method of FIG. 5, consistent with various aspects of the present disclosure.

FIG. 7 is a flowchart of a method of operating the electrosurgical system to puncture tissue, consistent with various aspects of the present disclosure.

FIGS. 8A-8C are a series of schematic diagrams of operations of crossing the atrial septum according to the method of FIG. 7, consistent with various aspects of the present disclosure.

FIGS. 9A and 9B are views of an alternative distal tip of the transseptal crossing member. More specifically, FIG. 9A is a cross-sectional view of the alternative distal tip as indicated by line 9-9 in FIG. 9B, consistent with various aspects of the present disclosure. FIG. 9B is a front view of the alternative distal tip, consistent with various aspects of the present disclosure.

FIG. 10 is a cross-sectional view of an alternative distal tip from an equivalent perspective to that of FIG. 9A, consistent with various aspects of the present disclosure.

FIG. 11 is a cross-sectional view of an alternative distal tip from an equivalent perspective to that of FIG. 9A, consistent with various aspects of the present disclosure.

FIG. 12 is a top view of an alternative distal tip, consistent with various aspects of the present disclosure.

FIG. 13 is a cross-sectional view of an alternative distal tip, consistent with various aspects of the present disclosure.

FIG. 14 is a partial cross-sectional view of an alternative distal tip, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

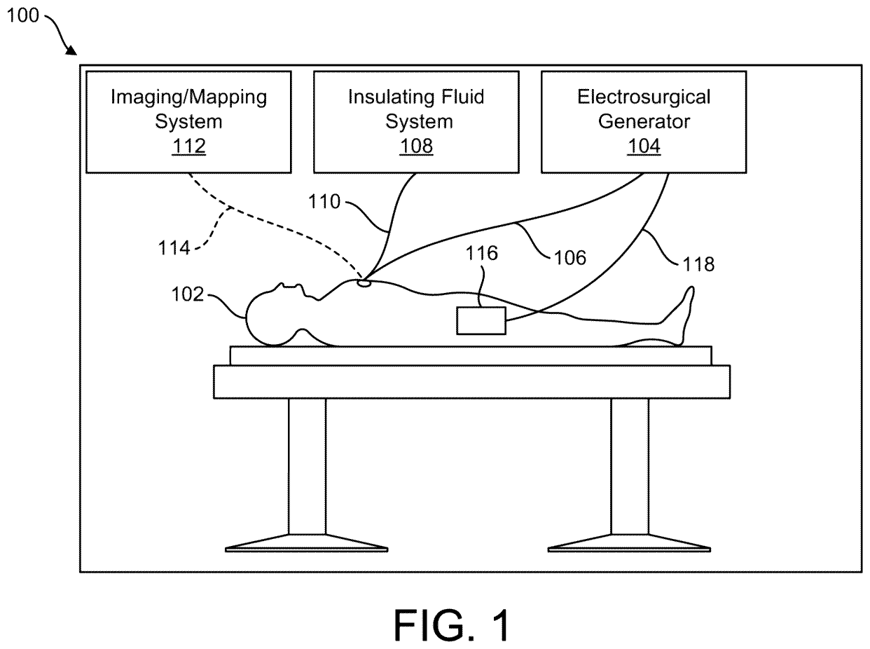

FIG. 1 shows an electrosurgical system 100 for treating a patient 102. In the illustrated embodiment, the system 100 includes an electrosurgical generator 104 with a transseptal crossing system 106, an insulating fluid system 108 with a delivery tube 110 connected to the transseptal crossing system 106, and an imaging/mapping system 112 for tracking the crossing system 106 in the patient 102. The imaging/mapping system 112 can use an external fluoroscopy system (not shown) and/or a mapping catheter 114 (shown in phantom) (such as, for example, the OPAL HDx™ mapping system from the Boston Scientific Corporation).

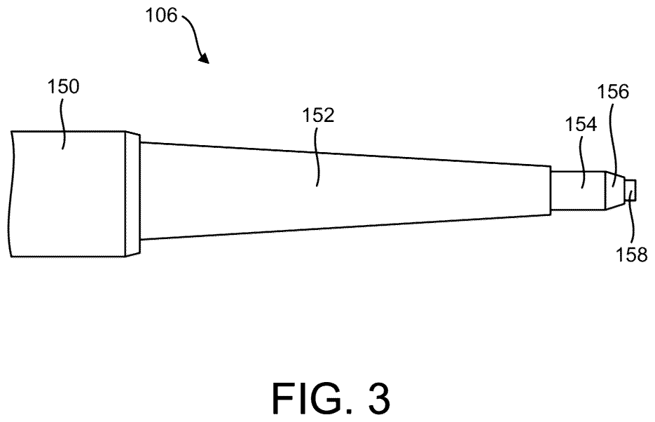

In the illustrated embodiment, the electrosurgical generator 104 is configured to provide energy, such as radiofrequency (RF) electrical energy, to the crossing system 106. Typically, the conductive crossing system 106 is permanently electrically insulated with the exception of a small distal portion, formed as a vaporizing electrode (shown in FIG. 3), that is intended to deliver the RF energy to the target tissue. To achieve tissue vaporization, the target tissue is rapidly heated. If heating is too slow, the tissue is desiccated rather than vaporized. Rapid heating is achieved through high current density at the electrode-tissue interface, meaning the electrode is either relatively small and/or the energy delivered is relatively high.

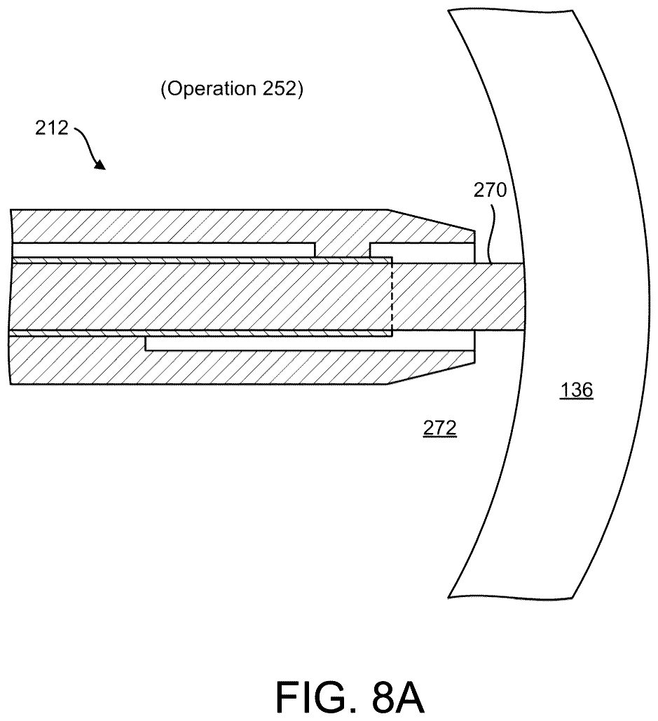

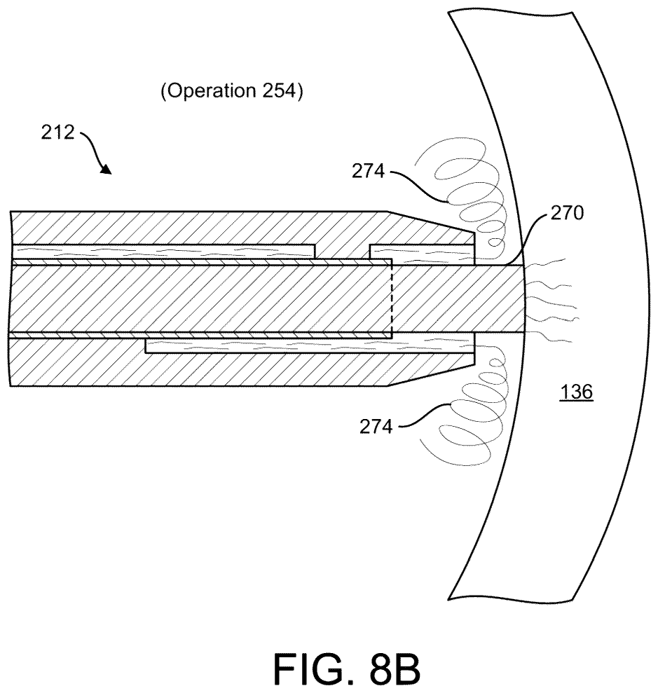

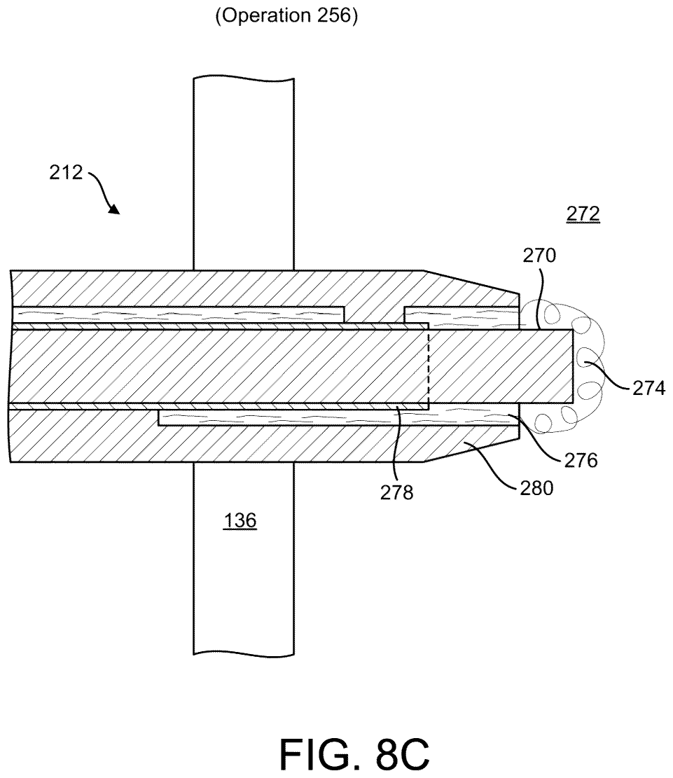

The delivery of energy to the target tissue can also heat the crossing system 106 itself. Thus, the electrical insulation around the crossing system 106 (especially near the electrode) should be able to withstand such heat without breaking down. While typically the insulation has been made from per-and polyfluoroalkyl substances (PFAS) (e.g., polytetrafluoroethylene (PTFE)), other materials may have the advantage of being less environmentally problematic. Such alternative materials, however, may lack the heat performance of the traditional materials, so the crossing system 106 should be designed to reduce and/or remove the heat generation within itself while still providing adequate heat generation in the target tissue. Therefore, the system 100 includes the insulating fluid system 108 that provides an electrically insulating fluid (e.g., dextrose liquid or carbon dioxide (CO₂) gas, shown in FIG. 8B) to surround the electrode at the distal end of the crossing system 106.

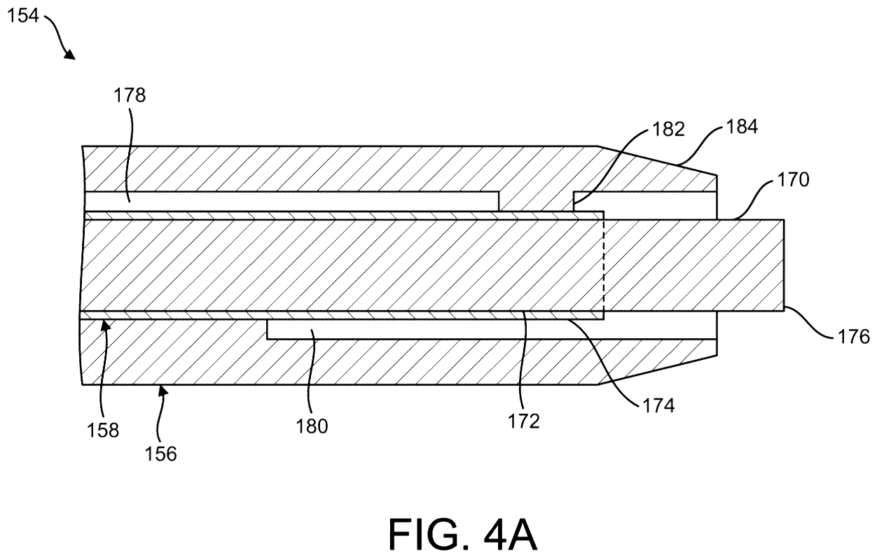

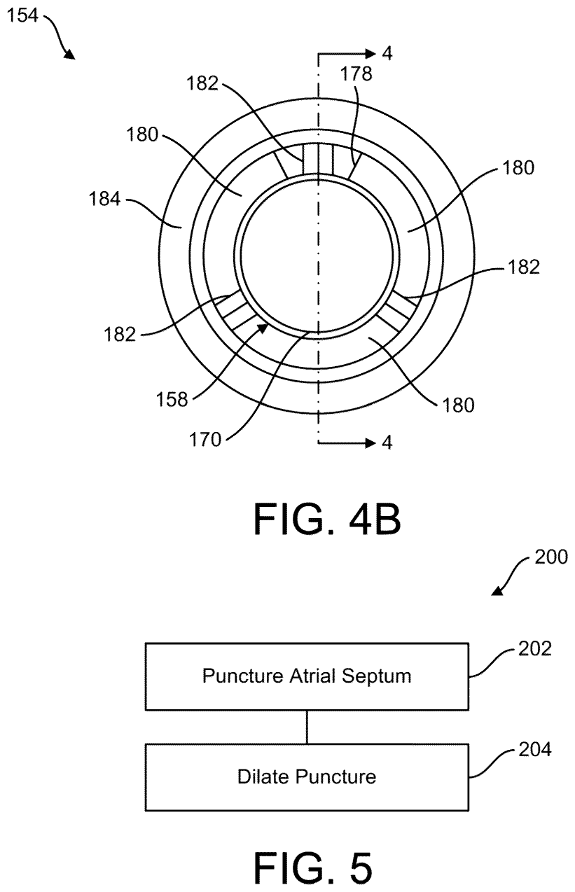

In the illustrated embodiment of FIG. 1, the crossing system 106 is monopolar and includes a single vaporizing electrode (i.e., an active electrode, shown in FIG. 4), so the system 100 also includes a patch electrode 116 (i.e., an indifferent or dispersive electrode). The patch electrode 116 has a large surface area to lower current density, so the patch electrode 116 is typically located on the back, buttocks, or upper leg of the patient 102, although there may be other suitable locations as well. The patch electrode 116 returns the RF electrical energy to the electrosurgical generator 104 through the lead 118. In some embodiments, the RF energy for a monopolar puncture function is provided by the electrosurgical generator 104 at a selected voltage and a continuous current (100% on, or 100% duty cycle). For example, if a power setting of 50 watts (W) is used for puncturing (which can mean that the instantaneous power is higher than 50 W), the voltage can range from approximately 164 volts (V) to 400 V root mean square (RMS).

In addition, the electrosurgical generator 104 can include a plurality of functions and provide programmed and custom settings via an interface (not shown). For example, the electrosurgical generator 104 provides RF energy to the crossing system 106 as an alternating current having a frequency in the range of 100 kilohertz (kHz) to 10 megahertz (MHz). Such puncturing RF energy can be applied in the form of a continuous waveform signal or in bursts of a waveform signal. In the latter case, the individual bursts of the waveform signal can have a duration of about 300 milliseconds (ms) with a rest interval between pulses of about 700 ms, although other durations of bursts and intervals can be used. In some embodiments, the waveform signals themselves can be sinusoidal or square waves that are bi-phasic. Furthermore, the electrosurgical generator 104 can be couplable to other electrosurgical tools and/or the electrosurgical generator 104 can receive signals (e.g., from the crossing system 106) to monitor the patient 102. In some embodiments, the electrosurgical generator 104 controls the insulating fluid system 108, so the initiation and cessation of RF energy and of the electrically insulating fluid can be synchronized (e.g., simultaneous).

The components and configuration of electrosurgical system 100 allow for target tissue to be vaporized. In some embodiments, the tissue vaporization allows the crossing system 106 to puncture through the atrial septum for treatment of the left side of the heart of the patient 102. While examples of the devices, systems, and methods of the present disclosure are presented in the context of a transeptal puncture, a person having ordinary skill in the art will recognize other applicable contexts. For example, the electrosurgical systems of the present disclosure can be employed to puncture a pericardium layer of a patient for epicardial access and/or to remove accumulation of atheromatous material on the inner walls of vascular lumens.

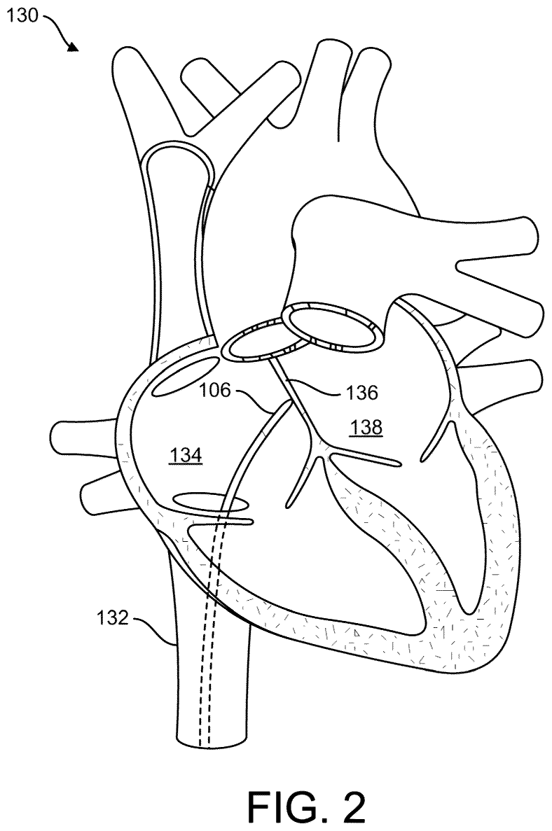

FIG. 2 shows a heart 130 of the patient 102 (shown in FIG. 1) with selected portions cut away. In the illustrated embodiment, the crossing system 106 extends through the inferior vena cava 132 from a surgical entry site (not shown) that is distal to the heart 130. The distal end of the crossing system 106 is positioned in the right atrium 134 and is in contact with the atrial septum 136. In the illustrated embodiment, the tip of the crossing system 106 is positioned at the fossa ovalis since this region of the atrial septum 136 is relatively safe and easy to puncture. Once the atrial septum 136 is crossed, the physician (not shown) will have access to the left atrium 138 (e.g., for treatment thereof).

In some use cases, such as a septal crossing, the electrode would be surrounded by a conductive liquid medium, such as blood, that is near the target tissue. When the crossing system 106 is operating in such an environment, any surface of the electrode that is in contact with the conductive liquid (instead of with the target tissue or the insulative fluid) provides a shunt path for the electrical current. These alternative electrical pathways do not help vaporize the target tissue, so they decrease the efficiency of the tissue vaporization process. Furthermore, these pathways can cause the blood to locally coagulate and form thrombotic material that can lead to embolization and subsequent deleterious health effects for the patient 102 (shown in FIG. 1). Therefore, components and methods to prevent such occurrences are discussed in the present disclosure.

FIG. 3 shows a distal end of the crossing system 106. In the illustrated embodiment, the crossing system 106 includes a sheath 150, a dilator 152, and a crossing device 154. The crossing device 154 is an elongated tissue vaporization device that can have the form of, for example, a wire, a needle, forceps, scalpels, or other devices that puncture and/or cut tissue. In some embodiments, a wire is a solid, stiff but elastically deformable member with a generally straight and/or helical configuration. In some embodiments, a needle is a hollow, flexible member with a generally straight configuration through which fluid can be pumped. The fluid can exit near an electrode that is positioned at the distal end of the needle, and the electrode can be connected to the electrosurgical generator 104 with a conductor since the flexible member can be made from an electrically insulating material. In some embodiments, forceps are a dual-levered instrument capable of grasping and/or holding tissue or other objects between their distal ends. In some embodiments, a scalpel is a bladed instrument with a sharpened edge capable of cutting tissue or other objects.

In some embodiments, the crossing system 106 has an overall length between about 55 centimeters (cm) and about 300 cm. The sheath 150 is an elongate member with a central lumen (not shown), in which the dilator 152 and the crossing device 154 are slidably positioned. The central lumen diameter is similar to the outer diameter of the majority of the dilator 152 (except for the distal tip), and the sheath 150 is tapered at the distal end to make the transition between the sheath 150 and the dilator 152 smoother. In addition, the sheath 150 can be a steerable sheath and/or have a fixed or adjustable curve at the distal end for positioning of the dilator 152 and the crossing device 154. In some embodiments, the sheath 150 and the dilator 152 are generally similar to those of the VersaCross™ Access Solution from Boston Scientific.

In the illustrated embodiment, the dilator 152 is an elongate member with a central lumen (not shown), in which the crossing device 154 is slidably positioned. The central lumen diameter is similar to the outer diameter of the crossing device 154, and the dilator 152 is tapered at the distal end to make the transition between the dilator 152 and the crossing device 154 smoother. In addition, the dilator 152 can have a fixed or adjustable curve at the distal end for positioning of the crossing device 154 against the atrial septum 136 (shown in FIG. 2).

In the illustrated embodiment, the crossing device 154 is an elongate member with an electrically insulating cover 156 over a central member 158. In some embodiments, a diameter of the crossing device 154 is between about 0.50 millimeters (mm) and about 1.0 mm. The cover 156 comprises, for example, parylene, polyimide, polyethylene terephthalate (PET), polyurethane, silicon dioxide (SiO₂), PTFE heat shrink, or combinations thereof. The central member 158 is an electrically conductive elongate member comprising stainless steel, nitinol, platinum, gold, iridium, or combinations thereof.

FIG. 4A shows the crossing device 154 with the central member 158 positioned inside of the cover 156. The central member 158 comprises an electrode 170, a central electrical conductor 172, and an insulative layer 174. The electrode 170 is positioned at the distal end of the central member 158, so the electrode 170 forms the distal end of the crossing device 154. The electrode 170 is electrically connected to the electrosurgical generator 104 (shown in FIG. 1) via the conductor 172. In some embodiments, the electrode 170 is an exposed section of the conductor 172, and in other embodiments, the electrode 170 is connected to the distal end of the central electrical conductor and has the same diameter as the insulative layer 174. The electrode 170 has a cylindrical shape with a flat distal end having a distal circular edge 176, thus the electrode 170 is atraumatic (i.e., not sharp). In other embodiments, however, the crossing device has a distal domed shape. In addition, the insulative layer 174 extends around the exterior of the conductor 172. In other embodiments where the cover 156 is electrically insulative per se, the insulative layer 174 is absent.

In the illustrated embodiment, a lumen 178 is a long void that is defined by the cover 156 and the central member 158. The lumen 178 is fluidly connected to the delivery tube 110 (shown in FIG. 1), so the lumen 178 receives electrically insulating fluid (shown in FIG. 8B) from the insulating fluid system 108 (shown in FIG. 1). The lumen 178 drains into a plenum 180 that begins proximal to the electrode 170 and overlaps a significant distance (e.g., at least halfway) with the electrode 170. In other embodiments, the plenum 180 does not overlap the electrode 170.

FIG. 4B shows that the plenum 180 extends around substantially all of the central member 158, although the cover 156 includes a plurality of supports 182 that extend through the plenum 180 to contact the central member 158. The plurality of supports 182 hold the central member 158 in the center of the plenum 180 to ensure even flow of the insulating fluid around the electrode 170. In some embodiments, the supports 182 have a symmetrical airfoil/hydrofoil shape that is designed to reduce impedance and disruption of the insulating fluid. While three supports 182 are shown in FIG. 4B, a greater or fewer number of supports can be used. As shown in FIG. 4A, the supports 182 are in contact with the insulative layer 174, so the supports 182 terminate proximally from the distal end of the electrode 170 to ensure that the insulating fluid covers the cylindrical exterior surface of the electrode 170. In some embodiments, the cover is an integral portion of the dilator, and in such embodiments, the supports are also integral portions of the dilator.

Returning to FIG. 4B, the lumen 178 subtends a sector (e.g., less than one quarter) of what would otherwise be the cover 156 proximate to the central member 158. Thereby, the lumen 178 is bordered by the cover 156 on three sides and the central member 158 on the remaining side. In other embodiments, the lumen 178 extends around substantially all of the central member 158 and/or the lumen 178 includes a separate tube (e.g., the delivery tube 110, shown in FIG. 1) that directs the flow of the insulating fluid through the crossing device 154 to the plenum 180. In addition, the cover 156 includes a chamfer 184 to reduce the distal end diameter of the cover 156 to match the diameter of the electrode 170 more closely.

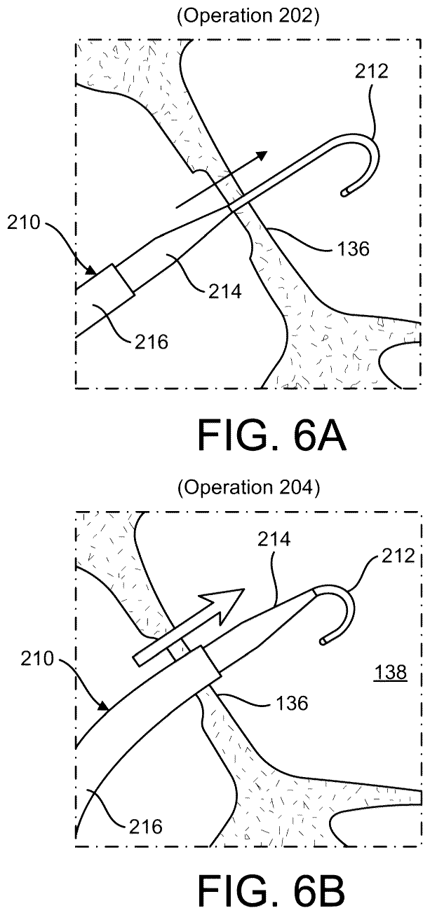

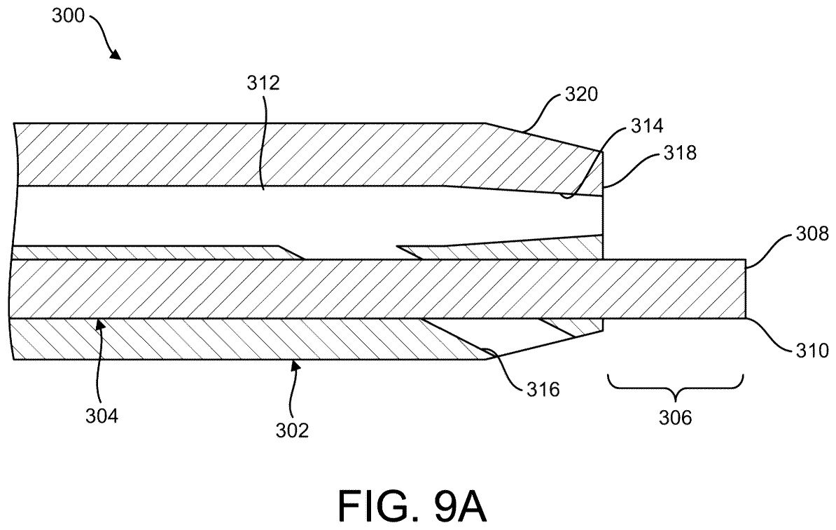

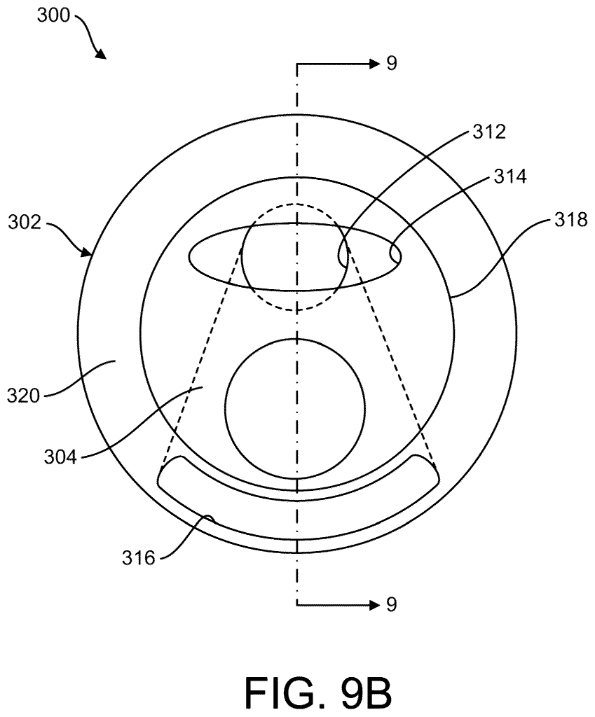

FIG. 5 shows a method 200 of crossing the atrial septum 136 using the crossing system 210 which can be the same as or similar to the crossing system 106 (shown in FIG. 2). FIGS. 6A and 6B show the operations of crossing the atrial septum 136. The distal tip of the crossing system 210 can be the same as or similar to the crossing device 154 (shown in FIG. 4C) or a different crossing device can be used (e.g., the crossing devices shown in FIG. 9A, 9B or 10). FIGS. 5 and 6A and 6B will now be discussed in conjunction with one another, and each operation of the method 200 is illustrated by a corresponding one of FIGS. 6A and 6B.

In the illustrated embodiment, the method 200 begins with a crossing system 210 being positioned near the atrial septum 136. At operation 202, as shown in FIG. 6A, the crossing device 212 is energized by the electrosurgical generator 104 while the insulating fluid system 108 is delivering insulating fluid to the distal end of the crossing device 212. Then, the physician applies distally-oriented force to the crossing device 212 (as indicated by the arrow). Thus, the crossing device 212 punctures through the atrial septum 136, and then the crossing device 212 is deenergized and the insulating fluid flow ceases (e.g., simultaneously). As shown in FIG. 6A, the crossing device 212 has a J-shape that is elastically deformed to be straight when the crossing device 212 is in the dilator 214, so the crossing device 212 returns to the J-shape when the crossing device 212 exits the dilator 214.

At operation 204, as shown in FIG. 6B, the physician applies distally-oriented force to the dilator 214 and to the sheath 216 (as indicated by the arrow). Thus, the dilator 214 and the sheath 216 follow the crossing device 212 through the atrial septum 136. As the dilator 214 follows the crossing device 212, the puncture in the atrial septum 136 is gently stretched by the dilator 214 so that the sheath 216 can enter the left atrium 138. Once the sheath 216 is positioned in the left atrium 138, one or more of the crossing device 212, the dilator 214, and the sheath 216 can be withdrawn and replaced with another component, such as, for example, a left atrial appendage closure (LAAC) implant device (not shown).

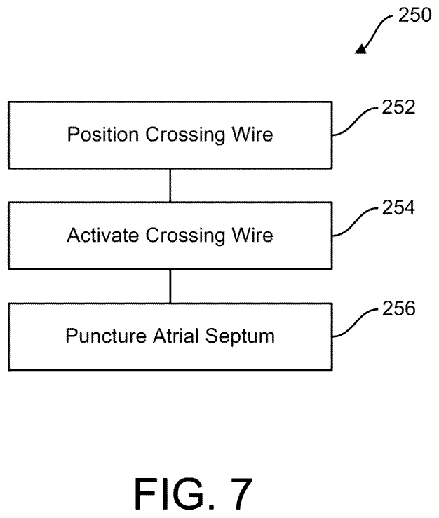

FIG. 7 shows a method 250 of puncturing the atrial septum 136. FIGS. 8A-8C show the operations of puncturing the atrial septum 136 using the crossing device 212. FIGS. 7 and 8A-8C will now be discussed in conjunction with one another, and each operation of the method 250 is illustrated by a corresponding one of FIGS. 8A-8C.

In the illustrated embodiment, the method 250 begins with operation 252 wherein the crossing device 212 is positioned in direct contact with the atrial septum 136. As shown in FIG. 8A, the distal end of the electrode 270 is pressed against the atrial septum 136, so the distal end of the electrode 270 is not in contact with the surrounding blood 272.

At operation 254, as shown in FIG. 8B, the physician has activated the insulating fluid system 108 first and then the electrosurgical generator 104 second. Activating the insulating fluid system 108 flows the insulating fluid 274 around the electrode 270. The insulating fluid 274 displaces the blood 272 that was previously in contact with the electrode 270. Thus, the cylindrical exterior of the electrode 270 is electrically insulated from the blood 272. Activating the electrosurgical generator 104 energizes the electrode 170. Virtually all of the RF energy being transmitted to the electrode 170 flows into the cells of the atrial septum 136 that are closest to the electrode 170, vaporizing the cells.

The concentration of the RF energy into solely the atrial septum increases the efficiency and ease of the crossing. In particular, the crossing device 212 penetrates through the atrial septum 136 with minimal tenting of the atrial septum 136 and with minimal jumping of the crossing device 212 upon breakthrough. The ease of the puncture means that the physician does not need to exert as much force on the crossing device 212 compared to a prior art crossing system that has inefficient or diffuse RF energy delivery. The inefficiency or diffuseness of the prior art can result from insufficient insulation of the electrode from the blood 272. Such an electrode would maintain contact with the blood 272 even when in contact with the atrial septum 136, so only a fraction of the RF energy provided to the electrode would be delivered to the atrial septum 136. The inefficient or diffuse delivery of the RF energy can result in the tissue cells receiving an insufficient amount of power for vaporization but enough for the tissue cells to be desiccated instead. Desiccation increases the electrical resistance of the affected tissue cells because there are fewer polar water molecules in the cells to conduct electricity, so the result is that even more power flows into the blood 272 instead of into the atrial septum 136.

At operation 256, as shown in FIG. 8C, the crossing device 212 has penetrated through the atrial septum 136. In some embodiments, the time to puncture the atrial septum 136 can be about 300 ms to about 400 ms. Some electrosurgical generators, however, will continue to deliver power to the electrode 270 due to their programming and/or user selection/input. The total “on” time of such a system can be, for example, multiple seconds(s) (e.g., 2 s to 5 s), which means that the electrode 270 is still powered well after the puncture has been completed. In such a scenario, the insulating fluid 274 that been insulating the cylindrical side of the electrode may have been wiped off of the electrode 270 as it passed through the atrial septum 136. But the insulating fluid 274 is still being pumped out of the plenum 276, and this new insulating fluid 274 can again displace all of the blood 272 around the electrode 270. Once the electrode 270 is electrically insulated on the cylindrical side and the distal side, the amount of current flowing through the electrode 270 will be reduced until cessation of power delivery by the electrosurgical generator 104 (shown in FIG. 1). On the other hand, a prior art electrode that does not include an insulative fluid system, after puncturing the atrial septum 136, could be generating and releasing gaseous bubbles and/or forming thrombotic material in the left atrium 138 of the heart 130 (shown in FIG. 2).

The components and configuration of the crossing device 212 and the bathing of the electrode 270 with the insulating fluid 274 requires less electrical power in the crossing device 212 than a prior art system without an insulating fluid system. Less power flowing through the electrode 270 means that there is less heat generated at the electrode 270, so less heat is being transferred to the insulative layer 278 and/or to the cover 280. Thus, the material used for the layer 278 and/or the cover 280 can be less heat resistant than in prior art crossing devices.

In an alternative embodiment to method 250, the order of the operations is different. For example, in some embodiments, the insulating fluid 274 is dispensed and electrode 270 is energized prior to the crossing device 212 contacting the atrial septum 136. In such an embodiment, the electrode 270 would be surrounded by a cloud of insulating fluid 274 that would be deformed as the distance between the electrode and the atrial septum closed. Eventually, the thickness of the insulating fluid 274 between the electrode 270 and the atrial septum 136 would no longer be sufficient, which would cause a dielectric breakdown of the insulating fluid 274. Thereby, the distal end of the electrode 270 and the atrial septum 136 would be electrically connected, and the nearby cells of the atrial septum 136 would be vaporized.

FIG. 9A shows an alternative crossing device 300 that includes a body 302 and a lead 304 that extends through the body 302 along an axis that is offset from the center of the body 302. The majority of the lead 304 is electrically insulated by the body 302, but the electrode portion 306 is uncovered. The electrode portion 306 is the section of the lead 304 that is positioned outside of the body 302 and forms the distal end of the crossing device 300. The electrode 306 has a cylindrical shape with a flat distal end 308 having a distal circular edge 310, thus the electrode portion 306 is atraumatic (i.e., not sharp). In other embodiments, however, the electrode has a distal domed shape. In addition, the lead 304 is electrically connected to the electrosurgical generator 104 (shown in FIG. 1).

In the illustrated embodiment, a lumen 312 is a long void that is defined by the body 302. The lumen 312 is fluidly connected to the delivery tube 110 (shown in FIG. 1), so the lumen 312 receives the electrically insulating fluid 274 (shown in FIG. 8C) from the insulating fluid system 108 (shown in FIG. 1). Proximal from the distal end of the body 302, the lumen 312 is connected to two separate exits—the inline path 314 and the transverse path 316. The inline path 314 extends straight from the lumen 312 and out to a distal end 318 of the body. The transverse path 316 extends from the lumen 312 at an angle such that the transverse path 316 crosses the lead 304 and extends out to a chamfer 320 of the body 302. Thus, the transverse path 316 exits the body 302 on the opposite side of the lead 304 from the lumen 312.

FIG. 9B shows that the inline path 314 transitions from the circular cross-section of the lumen 312 to an elliptical cross-section at the distal end 318. In addition, the cross-section of the transverse path 316 widens from the lumen 312 to the chamfer 320 so that the insulating fluid 274 can pass around the lead 304 prior to exiting the body 302. Having both the inline path 314 and the transverse path 316 on either side of the lead 304 allows the lead to be bathed in the insulating fluid 274 during the puncturing process. In some embodiments, flow control features (not shown) are positioned in one or both paths 312, 314 to balance the flow of the insulating fluid 274 to ensure coverage of the lead 304 by the insulating fluid 274. For example, airfoils/hydrofoils, baffles, and/or constructions can be positioned in paths 312, 314 as desired.

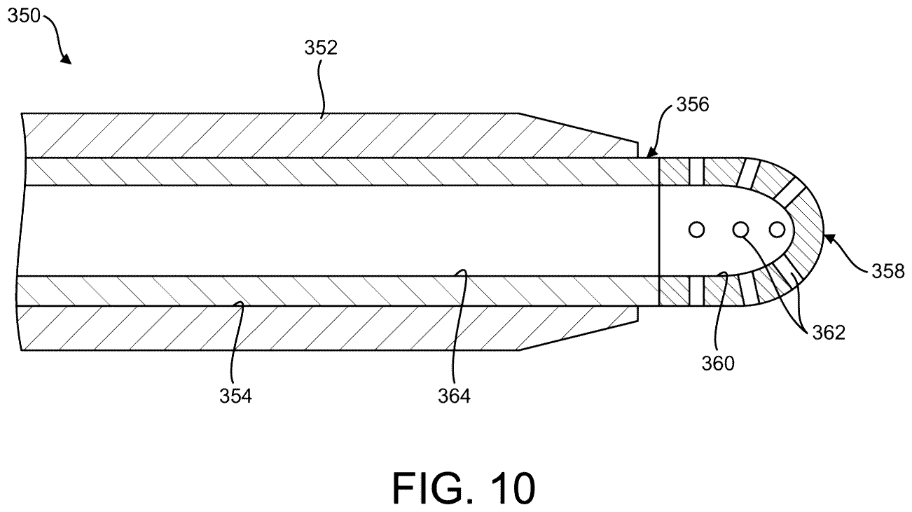

FIG. 10 shows an alternative crossing device 350 that includes a body 352 with a central lumen 354 and a hollow conductor 356 that extends through the body 352 inside of the lumen 354. In the illustrated embodiment, the crossing device 350 also includes an electrode 358 that is connected to the distal end of the hollow conductor 356. The electrode 358 is electrically connected to the electrosurgical generator 104 (shown in FIG. 1) via the conductor 356, to which the electrode 358 is connected to by way of, for example, by a welding, soldering, brazing, or adhesive. The electrode 358 has a hemispherical distal end and a cylindrical shape proximal therefrom, so the electrode 358 is atraumatic (i.e., not sharp).

In the illustrated embodiment, the electrode 358 includes an outer shell that defines a cavity 360, and there is an array of ports 362 that extend through the outer shell of the electrode 358. The hollow conductor 356 defines an interior path 364 for the insulating fluid 274 (shown in FIG. 8C) that is fluidly connected to the insulating fluid system 108 (shown in FIG. 1) and to the cavity 360. Thereby, the insulating fluid 274 can flow through the crossing device 350 and out of the electrode 358.

In the illustrated embodiment, each of the ports 362 is oriented orthogonally with respect to the exterior surface the electrode 358. In addition, the distal end of the electrode 358 does not include any ports 362. In some embodiments, for example, an area on the distal end of the electrode 358 that is defined by projecting a disc that is concentric with and has half of the radius of the cylindrical potion of the electrode 358 is free of any ports 362. Thereby, the distal end of the electrode 358 has a larger area of contact to vaporize the cells of the atrial septum 136 (shown in FIG. 8C) while the remaining surface area of the electrode 358 can be bathed in the insulating fluid 274 to be electrically insulated from the blood 272 (shown in FIG. 8C).

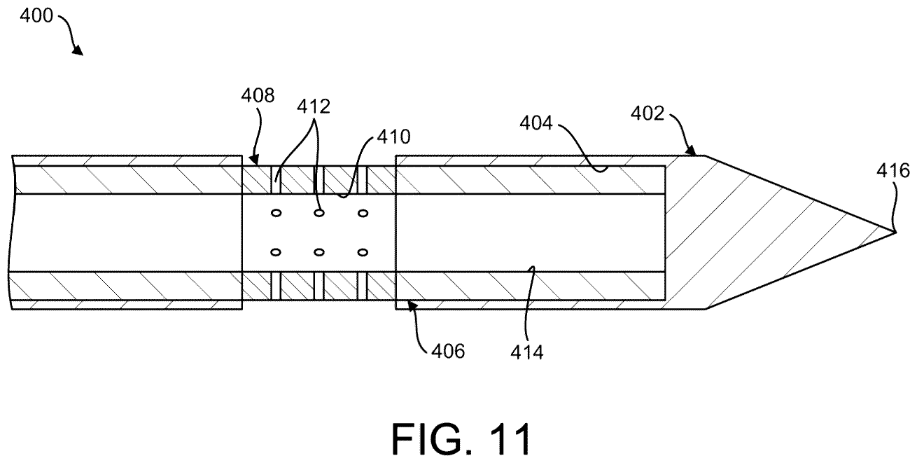

FIG. 11 shows a cross-section of a dissecting system 400 that includes a body 402 with a central lumen 404 and a hollow conductor 406 that extends through the body 402 inside of the lumen 404. In the illustrated embodiment, the body 402 electrically insulates the hollow conductor 406 from, for example, the blood 272 (shown in FIG. 8C). The dissecting system 400 also includes an electrode 408 that is positioned along the length of the hollow conductor 406, near the distal end. The electrode 408 is electrically connected to the electrosurgical generator 104 (shown in FIG. 1) via the hollow conductor 406, to which the electrode 408 is connected to by way of, for example, by a welding, soldering, brazing, or adhesive. In other embodiments, the electrode 408 is integral to the hollow conductor 406. In such embodiments, the electrode would be a portion of the hollow conductor that is not electrically insulated by the body 402.

In the illustrated embodiment, the electrode 408 has a hollow cylindrical shape with an outer shell that defines a cavity 410, and there is an array of ports 412 that extend through the outer shell of the electrode 408. In some embodiments, each of the ports 412 is oriented orthogonally with respect to the exterior surface the electrode 408. The hollow conductor 406 defines an interior path 414 for the insulating fluid 274 (shown in FIG. 8C) that is fluidly connected to the insulating fluid system 108 (shown in FIG. 1) and to the cavity 410. Thereby, the insulating fluid 274 can flow through the dissecting system 400 and out of the electrode 408.

In the illustrated embodiment, the body 402 includes a sharpened tip 416 at the distal end of the dissecting system 400. Thereby, a physician can puncture through tissue using the tip 416, and then position the dissecting system 400 such that the tissue is positioned adjacent to the electrode 408. Then the physician can activate the insulating fluid system 108 and the electrosurgical generator 104, and then sweep the dissecting system 400 through the tissue, creating a cut or slice in the tissue.

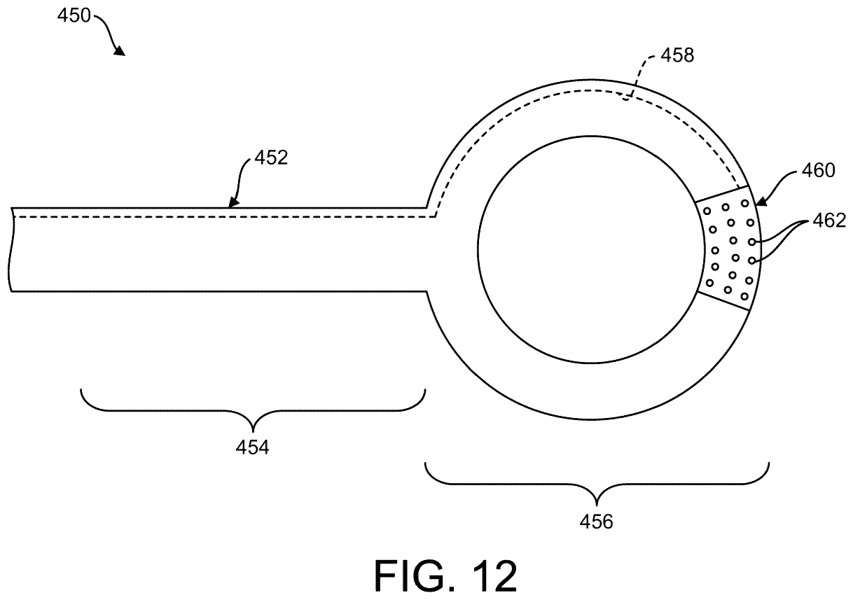

FIG. 12 shows a cutting system 450 that includes a body 452 having a straight portion 454 connected to a toroidal portion 456 at the distal end of the straight portion 454. The body 452 includes a lumen (not shown) positioned in the portions 452, 454, and a conductor 458 that extends through the body 452 along the wall of the lumen. In the illustrated embodiment, the cutting system 450 also includes a hollow electrode 460 that is connected to the distal end of the conductor 458. The hollow electrode 460 is electrically connected to the electrosurgical generator 104 (shown in FIG. 1) via the conductor 458, to which the hollow electrode 460 is connected to by way of, for example, by a welding, soldering, brazing, or adhesive. The hollow electrode 460 has a shape of a toroidal sector that matches the shape of the toroidal portion 456, so the hollow electrode 460 is atraumatic (i.e., not sharp).

In the illustrated embodiment, the hollow electrode 460 includes an outer shell that defines a cavity (not shown), and there is an array of ports 462 that extend through the outer shell of the hollow electrode 460. The lumen in the body 452 defines an interior path that is fluidly connected to the insulating fluid system 108 (shown in FIG. 1) and to the cavity in the hollow electrode 460. Thereby, the insulating fluid 274 (shown in FIG. 8C) can flow through the cutting system 450 and out of the hollow electrode 460. Moreover, the flow of the insulating fluid 274 enters both ends of the cavity in the hollow electrode 460, so the flow of the insulating fluid 274 is more uniform along the length of the hollow electrode 460.

In the illustrated embodiment, each of the ports 462 is oriented orthogonally with respect to the exterior surface the hollow electrode 460. The hollow electrode 460 can be positioned circumferentially opposite of the straight portion 454 (i.e., at the three o'clock position, as shown in FIG. 12), although in other embodiments, the hollow electrode 460 is positioned elsewhere in the toroidal portion 456 (e.g., at the six o'clock position). In some embodiments, flow control features (not shown) are positioned in the lumen of the toroidal portion 456 to balance the flow of the insulating fluid 274 to ensure coverage of the hollow electrode 460 by the insulating fluid 274. For example, airfoils/hydrofoils, baffles, and/or constructions can be positioned in the lumen as desired. Such features can be particularly advantageous in embodiments where the hollow electrode is positioned at a location other than directly opposite from the straight section 454.

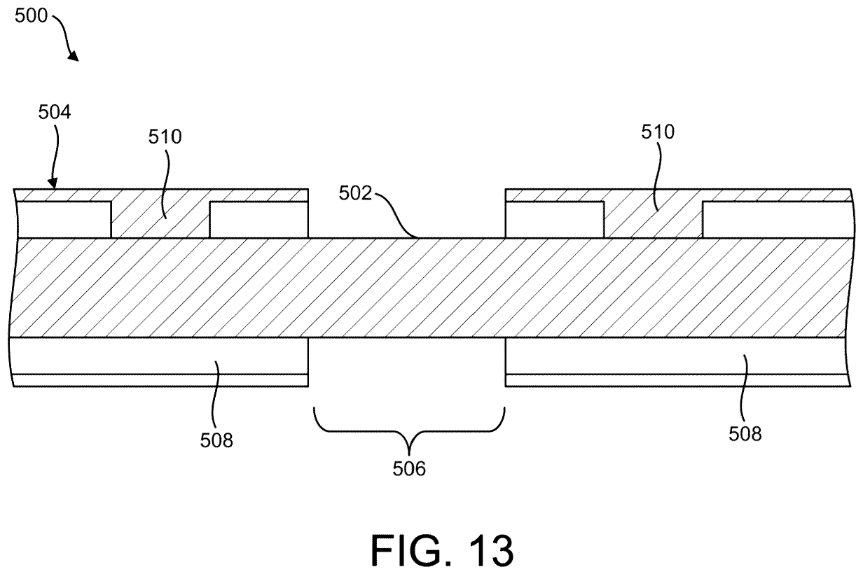

FIG. 13 shows the dissecting system 500 with a central member 502 positioned inside of a cover 504. The central member 502 is an electrically conductive device that is mostly electrically insulated by the cover 504, although in some embodiments, the central member 502 includes its own insulative layer (à la the insulative layer 174, shown in FIG. 4A). As shown, there is a gap 506 in the cover 504 that exposes a portion of the central member 502. The width of the gap 506 is sufficient to allow the central member 502 to contact the target tissue. In some embodiments, the dissecting system 500 has a similar overall shape (i.e., a straight portion with a distal toroidal portion extending therefrom) as the cutting system 450 (shown in FIG. 12), albeit with the gap 506 the place of the hollow electrode 460 (shown in FIG. 12). The central member 502 is electrically connected to the electrosurgical generator 104 (shown in FIG. 1).

In the illustrated embodiment, a lumen 508 is a long void that is defined by the cover 504 and the central member 502. The lumen 508 is fluidly connected to the delivery tube 110 (shown in FIG. 1), so the lumen 508 receives the electrically insulating fluid 274 (shown in FIG. 8B) from the insulating fluid system 108 (shown in FIG. 1). During operation of the dissecting system 500, the insulating fluid 274 flows into both sides of the gap 506 from the lumen 508 to displace any blood 272 (shown in FIG. 8C) that is in the vicinity of the central member 502.

In the illustrated embodiment, the lumen 508 extends around substantially all of the central member 502, although the cover 504 includes a plurality of supports 510 that extend through the lumen 508 to contact the central member 502 on either side of the gap 506, respectively. The supports 510 hold the central member 502 in the center of the lumen 508 to ensure even flow of the insulating fluid around the central member 502, especially the portion in the gap 506. In some embodiments, the supports 510 have a symmetrical airfoil shape that is designed to reduce impedance and disruption of the insulating fluid 274. The two sets on either side of the gap 506 can have any appropriate number of the supports 510. For example, there can be three supports 510 in each set (à la the supports 182, shown in FIG. 4B). In other embodiments, however, one or both sets of supports are not present, for example, in embodiments where the cover has two parts that originate from two separate catheters with the central member spanning therebetween.

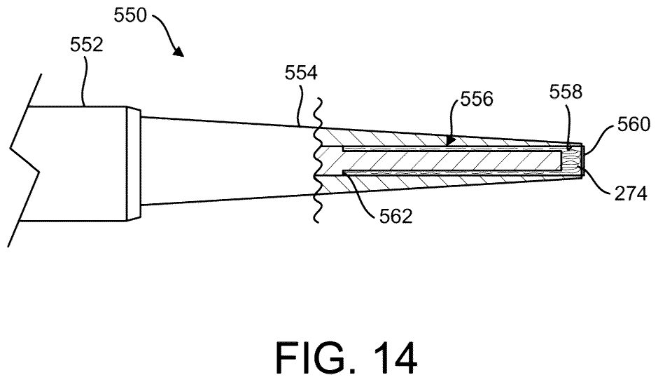

FIG. 14 shows the distal end of crossing system 550 in cross-section. In the illustrated embodiment, the crossing system 550 includes a sheath 552, a dilator 554, and a crossing device 556. The sheath 552 is an elongate member with a central lumen (not shown), in which the dilator 554 and the crossing device 556 are slidably positioned. The central lumen diameter is similar to the outer diameter of the majority of the dilator 554 (except for the distal tip), and the sheath 552 is tapered at the distal end to make the transition between the sheath 552 and the dilator 554 smoother. In addition, the sheath 552 can be a steerable sheath and/or have a fixed or adjustable curve at the distal end for positioning of the dilator 554 and the crossing device 556. In some embodiments, the sheath 552 and the dilator 554 are generally similar to those of a VersaCross™ Access Solution from Boston Scientific.

In the illustrated embodiment, the dilator 554 is an elongate member with a central lumen 558, in which the crossing device 556 is slidably positioned. The dilator 554 is tapered at the distal end to make the transition between the dilator 554 and the crossing device 556 smoother. In addition, the dilator 554 can have a fixed or adjustable curve at the distal end for positioning of the crossing device 556 against the atrial septum 136 (shown in FIG. 8C).

The diameter of the central lumen 558 is similar to the outer diameter of the crossing device 556, except at the distal end. The distal end of the dilator 554 functions as the cover for the crossing device 556 (in contrast with previous illustrated embodiments). There is a gap between the central lumen 558 and the crossing device 556 at the distal end of the dilator 554. This gap is pre-filled with insulating fluid 274, which is sealed in the dilator 554 between a cap 560 on the distal end and a shoulder 562 of the crossing device 556 on the proximal end. During operation of the crossing system 550, the crossing device 556 is advanced to expose the distal end to the atrial septum 136. In doing so, the cap 560 is pierced or otherwise displaced by the crossing device 556 while the shoulder 562 forces the insulating fluid 274 out of the end of the dilator 554. The insulating fluid 274 can then displace any blood 272 (shown in FIG. 8C) in the vicinity, which electrically insulates the crossing device 556 and prevents the shunting of RF energy into the blood 272.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical system for piercing tissue, the electrosurgical system comprising:
an electrosurgical generator configured to generate radiofrequency (RF) energy;
a fluid system configured to deliver electrically insulating fluid; and
a crossing member connected to the electrosurgical generator and the fluid system, the crossing member including:
an electrode positioned at a distal tip of the crossing member; and
a first fluid port positioned adjacent to the electrode, the first fluid port configured to direct the electrically insulating fluid around the electrode.

2. The electrosurgical system of claim 1, wherein:
the crossing member comprises a cover surrounding and spaced apart from a longitudinal portion of the electrode; and
the first fluid port is defined by the cover and the electrode.

3. The electrosurgical system of claim 1, wherein:
the crossing member further comprises a lumen connected to the first fluid port and a second fluid port; and
the electrode is positioned between the first fluid port and the second fluid port.

4. The electrosurgical system of claim 1, wherein the electrically insulating fluid is dextrose.

5. The electrosurgical system of claim 1, wherein the electrically insulating fluid is carbon dioxide.

6. The electrosurgical system of claim 1, wherein the crossing device is a wire.

7. The electrosurgical system of claim 1, wherein the crossing device is a needle.

8. An electrosurgical system for piercing tissue, the electrosurgical system comprising:
an electrosurgical generator configured to generate radiofrequency (RF) energy;
a fluid system configured to deliver electrically insulating fluid; and
a crossing member connected to the electrosurgical generator and the fluid system, the crossing member including:
an electrode positioned at a distal tip of the crossing member; and
a first fluid port that extends through the electrode, the first fluid port configured to direct the electrically insulating fluid around the electrode.

9. The electrosurgical system of claim 8, wherein the first fluid channel is one of an array of fluid channels positioned around the electrode but not at the distal tip.

10. The electrosurgical system of claim 8, wherein the crossing device is a wire.

11. The electrosurgical system of claim 8, wherein the crossing device is a needle.

12. The electrosurgical system of claim 8, wherein the electrically insulating fluid is dextrose.

13. The electrosurgical system of claim 8, wherein the electrically insulating fluid is carbon dioxide.

14. An electrosurgical system for piercing tissue, the electrosurgical system comprising:
an electrosurgical generator configured to generate radiofrequency (RF) energy;
a fluid system configured to deliver electrically insulating fluid; and
a crossing member connected to the electrosurgical generator and the fluid system, the crossing member including:
an electrode positioned along an exterior of the crossing member proximal from a distal tip; and
a first fluid channel that extends through an interior of the crossing member, the first fluid channel being fluidly connected to a fluid port configured to direct the electrically insulating fluid around the electrode.

15. The electrosurgical system of claim 14, wherein the distal tip of the crossing member is configured to puncture tissue.

16. The electrosurgical system of claim 14, wherein the first fluid channel is one of an array of fluid channels positioned around the electrode.

17. The electrosurgical system of claim 14, wherein the crossing device is a wire.

18. The electrosurgical system of claim 14, wherein the crossing device is a needle.

19. The electrosurgical system of claim 14, wherein the electrically insulating fluid is dextrose.

20. The electrosurgical system of claim 14, wherein the electrically insulating fluid is carbon dioxide.
